(19) United States
(12) Patent Application Publication
(10) Pub. No.: US 2002/0013564 A1
(43) Pub. Date: Jan. 31, 2002

(54) Perforated feminine sanitary protection device

(76) Inventors: Richard William Kubalek, Appleton, WI (US); Susan Jean Moder, Appleton, WI (US); Thomas Harold Roessler, Menasha, WI (US)

Correspondence Address: Karl V. Sidor, Kimberly-Clark Worldwide, Inc., 401 North Lake Street, Neenah, WI, 54957-0349, US

(21) Appl. No.: 09/923,032

(22) Filed: Aug. 6, 2001

Related U.S. Application Data
(63) Continuation-in-part of application No. 09/473,385, filed on Dec. 28, 1999, now abandoned.

(51) Int. Cl. A61F 13/15

(52) U.S. Cl. 604/385.01

(57) ABSTRACT

A feminine sanitary protection device includes an outer cover containing a panty shield and a vaginal insertion device. The outer cover and the panty shield are tri-folded over the vaginal insertion device, and joining elements join the side sections of the outer cover together. There are frangible lines in the side sections for separating portions of the outer cover in order to provide access to the panty shield and vaginal insertion device. The method of making the feminine sanitary protection device includes providing a continuously moving layer of material, forming frangible lines in the side sections of the material, applying a joining element in each of the side sections of the material, placing a plurality of panty shield components on the layer of material and joining the components to the layer of material to form panty shields, forming a plurality of individual panty shields and respective individual portions of the layer of material, and then folding each individual portion of the layer of material and its panty shield together.

### BACKGROUND OF THE INVENTION

The present invention relates to a feminine sanitary protection device that protects a user by absorbing and containing menstrual fluids and other body exudes as well as a method for making the same. More specifically, this invention relates to a feminine sanitary protection device which provides substantially complete sanitary protection, ease in handling, and discretion in packaging appearance as well as a method for making the same.

Absorbent articles are designed to absorb body fluids, including menses, and may come in different functional designs. In one design category, sanitary napkins are externally worn about the pudendal area and are designed primarily for heavy flow. In another category, panty liners or panty shields are thin products externally worn about the pudendal area and are designed for light flow. In yet another category, tampons are designed to be positioned internally within the vagina.

Sanitary napkins can have high absorptive capacity with either a thin or thick absorptive element. However, compressive forces of the wearer's thighs and pudendal region during any physical movement, such as walking, can cause the sanitary napkin to shift from an original position protecting the vulva area. After a relatively short period of time, the sanitary napkin may move away from the vaginal orifice. The wearer's movement, particularly vigorous movement such as rapid walking or running, also can cause discomfort such as by rubbing or chafing in the sensitive vulva area.

In addition to concerns of sanitary napkin movement and discomfort, a concern of high degree of wearing awareness is present. Some thick sanitary napkins have a high profile appearance when viewed through a wearer's outer garments. The sanitary napkins can be very apparent when worn with tight fitting clothing including slacks, body suits, swimming suits, or similarly thin or close fitting outer garments.

Panty liners or panty shields have been developed for light or low menstrual flows. Some panty liners or panty shields have the same concerns associated with sanitary napkins, although their thin profile makes them more flexible, less noticeable in appearance, and generally more comfortable than the bulky sanitary napkins. However, the thin profile panty liners or panty shields can have a drawback in the performance area of absorptive capacity.

Tampons are worn internally within the vaginal canal to intercept body fluid. Sometimes tampons may not function completely to prevent leakage because radial expansion of the tampon within the vaginal canal does not form a perfect seal. Yet without such radial expansion and swelling of the tampon within the vaginal canal, the tampon does not serve as a completely reliable sanitary protection device.

In an attempt to address the above stated problems, a sanitary napkin or a panty liner is rolled about a tampon or similar device, and then inserted into a pouch. However, additional problems arise associated with a napkin or panty liner rolled about a tampon or other similar device. One of these problems is that the napkin or panty liner can tend to curl after removal from the pouch, thereby making it more difficult, if not impossible, to adhere the curled napkin or panty liner onto an undergarment.

Another problem associated with a rolled napkin or panty liner is the loss of comfort and absorbency due to the undesired shape of a curled or partially curled napkin or panty liner.

Still another problem associated with a rolled napkin or panty liner is the potential loss of the embossing pattern on the cover, or the loss of some other cover characteristic.

### SUMMARY OF THE INVENTION

In response to the discussed difficulties and problems encountered in the prior art, a feminine sanitary protection device as well as a method for making such a device has been discovered.

In one form of the present invention there is provided a feminine sanitary protection device comprising an outer cover having a central longitudinal axis and including a top edge, a bottom edge, a pair of side edges, a first side portion between one of the side edges and the central longitudinal axis, and a second side portion between the other of the side edges and the central longitudinal axis. Absorbent device components (e.g., a liquid permeable liner and an absorbent medium) are joined to the outer cover between the first side portion and the second side portion to form an absorbent article such as, for example a panty shield. A first frangible line is in the first side portion, and a second frangible line is in the second side portion. The outer cover and the absorbent device (e.g., panty shield) are tri-folded so the outer cover forms the exterior of the device. Upon removal of portions of the outer cover outboard the frangible lines and unfolding the device, a remaining portion of the outer cover (to which the absorbent device components are joined) is adapted to for a liquid impermeable layer of the absorbent device (e.g., panty shield).

In another form of the present invention there is provided a feminine sanitary protection device comprising an outer cover having a central longitudinal axis and including a top edge, a bottom edge, a pair of side edges, a first side portion between one of the side edges and the central longitudinal axis, and a second side portion between the other of the side edges and the central longitudinal axis. Absorbent device components are joined to outer cover to form an absorbent article, and the outer cover and the absorbent article are folded together so the outer cover forms the exterior of the folded article. There is a mechanism in the first side portion and in the second side portion for removing at least a portion of the first side portion and at least a portion of the second side portion from the outer cover. Upon removal of at least a portion of the first side portion and at least a portion of the second side portion from the outer cover and unfolding the device, a remaining portion of the outer cover (to which the absorbent article components are joined) is adapted to for a liquid impermeable layer of the absorbent article.

In yet another aspect of the present invention there is provided a method for making a feminine sanitary protection device comprising the steps of:

providing a continuously moving layer of liquid impermeable material including a pair of side edges and a pair of side portions;

aligning a plurality of absorbent article components at spaced apart intervals with the continuously moving layer of liquid impermeable material and joining the components to the liquid impermeable material to form absorbent articles;

forming a frangible line in each of the side portions;

applying a joining element in each of the side portions;

forming a plurality of individual absorbent articles with its respective individual portions of the layer of liquid impermeable material, and

folding each individual portion of the layer of material and its absorbent article together.

In an embodiment, the plurality of absorbent article components may be supported on a carrier sheet that forms the bodyside cover or liner of the absorbent article. These steps may be carried out in sequence or may be carried out in a different order. For example, it is contemplated that the step of forming the frangible lines in each side portion may be carried out after the individual article is folded.

In another aspect of the present invention there is provided a method for making a feminine sanitary protection device comprising the steps of (a) providing a continuously moving layer of liquid impermeable material including a pair of side edges and a pair of side portions, (b) forming a frangible line in each of the side portions, (c) applying a joining element in each of the side portions, (d) placing a plurality of absorbent article components at spaced apart intervals on the continuously moving layer of liquid impermeable material and joining the components to the liquid impermeable material to form panty shields, (e) forming a plurality of individual absorbent articles and respective individual portions of the layer of material, and (f) folding each individual portion of the layer of material and its absorbent article together.

In still another aspect of the present invention there is provided a method of making a feminine sanitary protection device comprising the steps of (a) providing a continuously moving layer of liquid impermeable material including a pair of side edges and a pair of side portions, (b) forming a frangible line in each of the side portions, (c) placing a plurality of absorbent article components at spaced apart intervals on the continuously moving layer of liquid impermeable material and joining the components to the liquid impermeable material to form absorbent articles, (d) forming a plurality of individual absorbent articles and respective individual portions of the layer of material, and (e) folding each individual portion of the layer of material and its absorbent article together.

### BRIEF DESCRIPTION OF THE DRAWINGS

The above-mentioned and other features of the present invention and the manner of attaining them will become more apparent, and the invention itself will be better understood by reference to the following description of the invention, taken in conjunction with the accompanying drawings wherein:

FIG. 1 illustrates an elevational view of a feminine sanitary protection device,

FIG. 2 illustrates an exploded perspective of the device of FIG. 1,

FIG. 3 illustrates a perspective view of the device of FIG. 1 in a partially tri-folded form,

FIG. 4 illustrates a perspective view of a pouch containing the fully tri-folded device of FIG. 3,

FIG. 5 illustrates an elevational view of a panty shield with a vaginal insertion device different from FIG. 1,

FIG. 6 illustrates an elevational view of a panty shield with a vaginal insertion device different from FIG. 5, and

FIG. 7 illustrates one exemplary method of making a feminine sanitary protection device.

FIG. 8 illustrates another exemplary method of making a feminine sanitary protection device.

### DETAILED DESCRIPTION

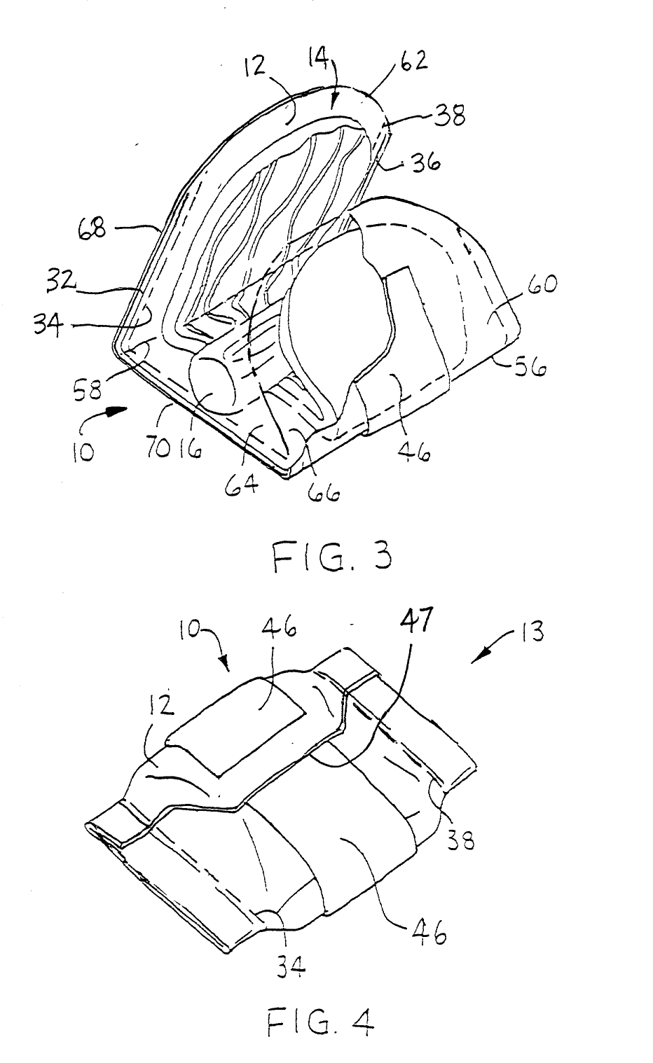

The feminine sanitary protection device of the present invention provides an absorbent article (e.g., panty liner or panty shield) constructed and folded in such manner that its outer cover is also adapted to function as a liquid impermeable layer of the absorbent article during use. The absorbent article may be folded over one or more vaginal insertion devices containing absorbents or medical devices for easy handling and use. Of course, the present invention is not limited to panty liners or panty shields and should be understood as encompassing any type of foldable absorbent article, hygienic cleansing article or medical device alone or in combination with one or more vaginal insertion devices. The term “folded” refers, by way of example only, to a C-fold or to a tri-fold as illustrated in FIG. 3 and may include other types of folds. The term “folded over” or variations thereof means the article (e.g., liner or shield) may have, for example, another article such as a tampon positioned within the folds. The term “vaginal insertion device” refers to a vaginal insert or vaginal insert applicator. By “vaginal insert”, it is meant, by way of example only, a tampon or vaginal medicinal insert such as a vaginal suppository. By “vaginal insert applicator”, it is meant, by way of example only, a tampon applicator or a vaginal medicinal insert applicator such as a vaginal suppository applicator.

In one aspect, the device of the present invention provides a more discreet, convenient, and portable option than carrying a absorbent article such as, for example, a panty shield and a vaginal insertion device separately. An integral pouch, made with biodegradable and/or non-biodegradable materials, serves to protect the panty shield (and vaginal insertion device—if included) prior to wear, and acts as a packaging agent. The present invention further provides efficiencies in material and manufacturing costs, and ease in consumer handling.

One advantage of the present invention is that it conveniently contains the products a woman needs to feel fresh and completely protected from stains on her undergarments or adjacent clothing. The present invention also provides a woman with an almost zero chance of experiencing staining on her undergarments when using these products or devices together.

Another advantage of the present invention is that it is more discreet and convenient than carrying two or more separate products. Thus, a woman does not need to go out and buy two or more separate products, thereby saving money and time. Nor does she need to carry them around separately, and make sure when it comes time to use them that all of the separate products still are available. The pouch keeps them fresh and protected from contamination.

Referring now to the drawings, common elements in all of the drawings are referenced using the same identifying numerals.

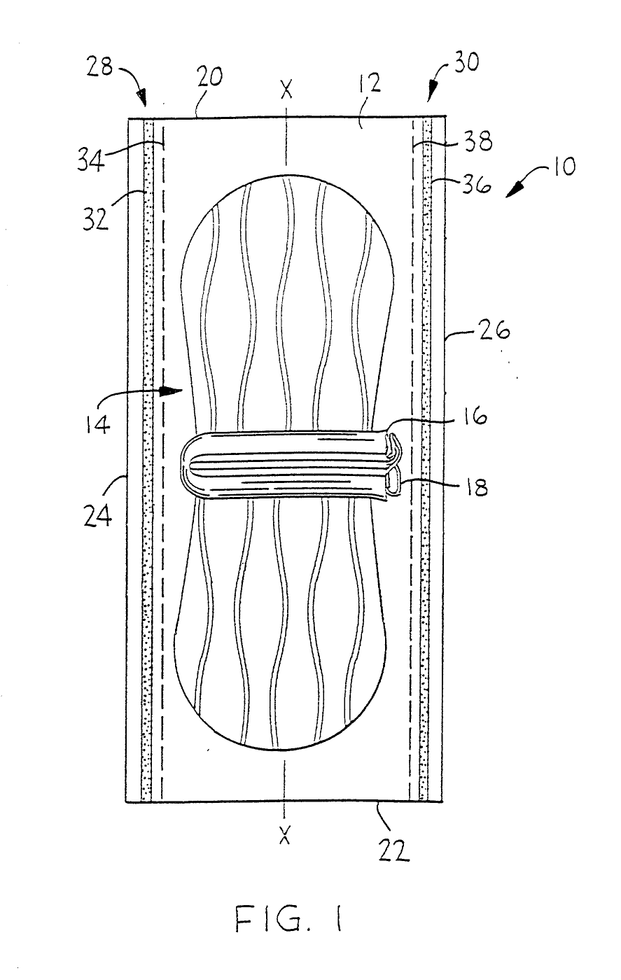

Referring now to FIGS. 1-3, a feminine sanitary protection device 10 of the present invention includes an outer cover 12, an integral absorbent article (e.g., panty shield) 14, and a vaginal insertion device, such as a tampon 16 including a withdrawal string 18. The absorbent article (hereinafter frequently referred to as a “panty shield”) 14 represents a sanitary napkin, a panty liner, or any other similar article designed to be worn by a woman. Alternatively and/or additionally, the absorbent article may represent any type of external or internal absorbent device, hygienic cleansing article or wipe and/or medical device. Panty shield 14, as illustrated in FIG. 1, has an hour glass shape, but it is understood that it can have any other suitable shape such as an oval, straight-sided, racetrack, or the like. A central longitudinal axis X-X can represent both the longitudinal centerline for outer cover 12 and panty shield 14. Tampon 16 is illustrated as being placed on panty shield 14 in an orientation generally perpendicular to that of central longitudinal axis X-X.

Outer cover 12 includes a top edge 20, a bottom edge 22, and a pair of side edges 24, 26 extending between edges 20, 22. The shape of outer cover 12 may be generally rectangular, but can have any other desired shape, such as an oval shape, racetrack shape, or the like. For ease of manufacturing, outer cover 12 is generally rectangular in shape. Outer cover 12 further includes a first side section 28 extending between top edge 20 and bottom edge 22, and from side edge 24 to central longitudinal axis X-X. Similarly, a second side section 30 extends between top edge 20 and bottom edge 22, and from side edge 26 to central longitudinal axis X-X. Within first side section 28 are a first joining element 32 and a first frangible line 34. First frangible line 34 is located between first joining element 32 and central longitudinal axis X-X. Similarly, second side section 30 includes a second joining element 36 and a second frangible line 38. Second frangible line 38 is between second joining element 36 and central longitudinal axis X-X. For purposes of economy, since first side section 28 with first joining element 32 and first frangible line 34 is identical to second side section 30 with second joining element 36 and second frangible line 38, only a description of first side section 28, first joining element 32, and first frangible line 34 will be made. It is understood then that this same description would apply to second side section 30, second joining element 36, and second frangible line 38.

First frangible line 34 provides a means or mechanism for removing at least a portion of first side section 28 from outer cover 12. The specific purpose for this means or mechanism for removing a portion of outer cover 12 will be explained hereafter. Thus, the purpose of first frangible line 34 is to allow that portion of outer cover 12 between first frangible line 34 and side edge 24 to be removed or torn along first frangible line 34. Frangible line 34 may, for example, be provided by partially cutting or otherwise thinning through the layer of material of which outer cover 12 is made. Alternatively and/or additionally, the frangible line 34 may be provided by a selected pattern of perforations and/or may be provided by a desired pattern of stress-fatigue weakening of outer cover 12, or the like. As illustrated, frangible line 34 is provided by a line of perforations in which there can be approximately 2-10 perforations per lineal inch.

First joining element 32 can be any suitable means or mechanism for joining respective portions of outer cover 12 together, as will be explained in greater detail hereafter. For example, first joining element 32 can be an adhesive applied along first side section 28, a thermal bondline, an ultrasonic bondline, or any other suitable means or mechanism for joining outer cover 12 together. If desired, for purposes of economy and manufacturing efficiency, first joining element 32 can be eliminated, and the means or mechanism for joining portions of outer cover 12 together can be provided by first frangible line 34. If first frangible line 34 is used in place of first joining element 32, it will serve both purposes of providing a joining of portions of outer cover 12 together, and providing for the removal of at least a portion of side section 28.

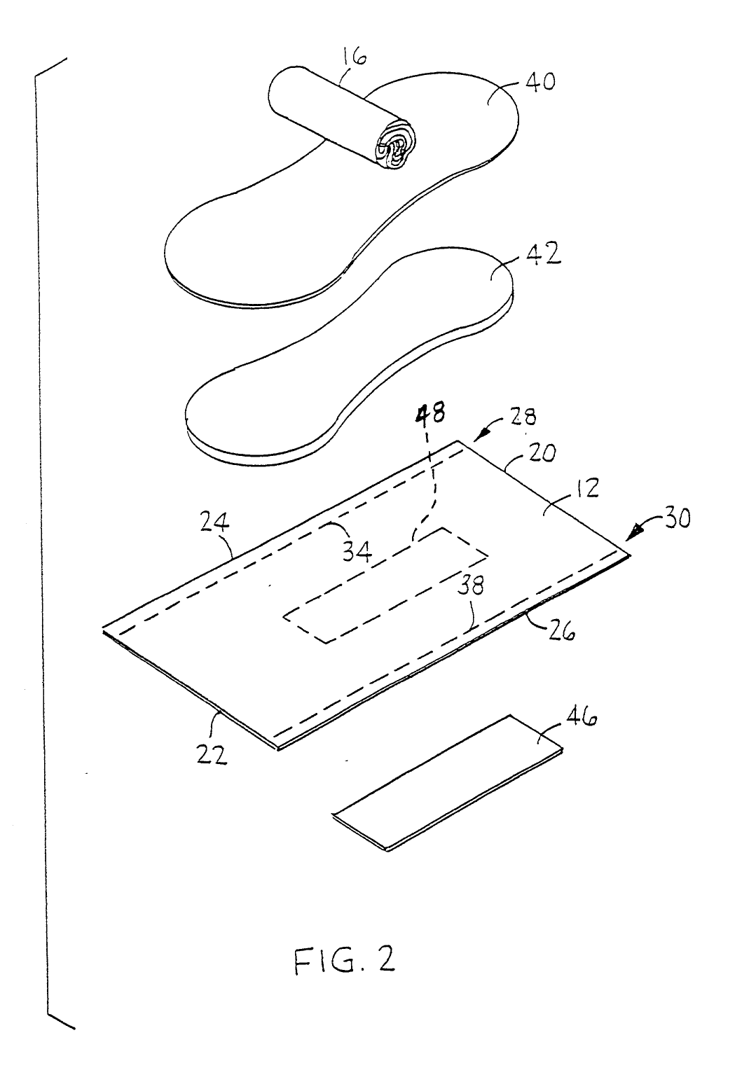

Referring now to FIG. 2, feminine sanitary protection device 10 of FIG. 1 is illustrated in an exploded, perspective view. Panty shield 14 generally comprises a liquid permeable cover 40, an absorbent medium 42, a liquid impermeable baffle which is the outer cover 12, and a release strip 46 that is removably adhered to outer cover 12 by release strip adhesive 48. Tampon 16, representing one type of vaginal insertion device, is also illustrated as being placed on cover 40 of panty shield 14.

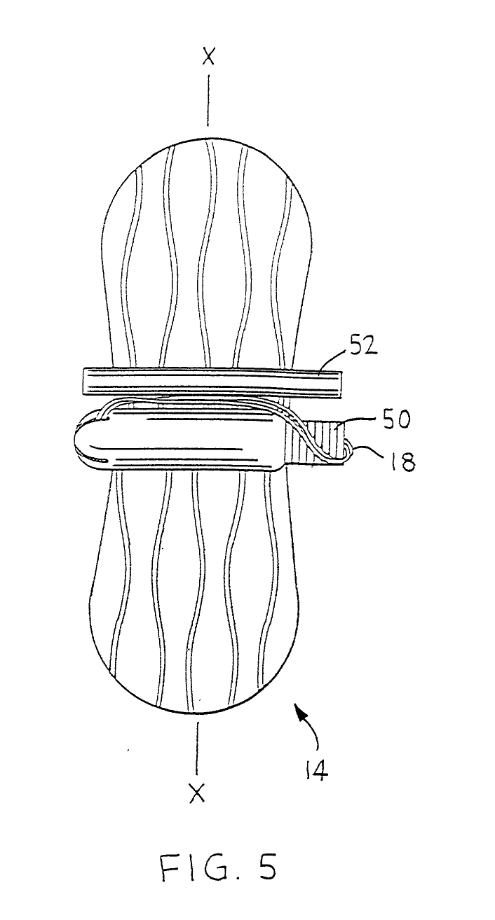

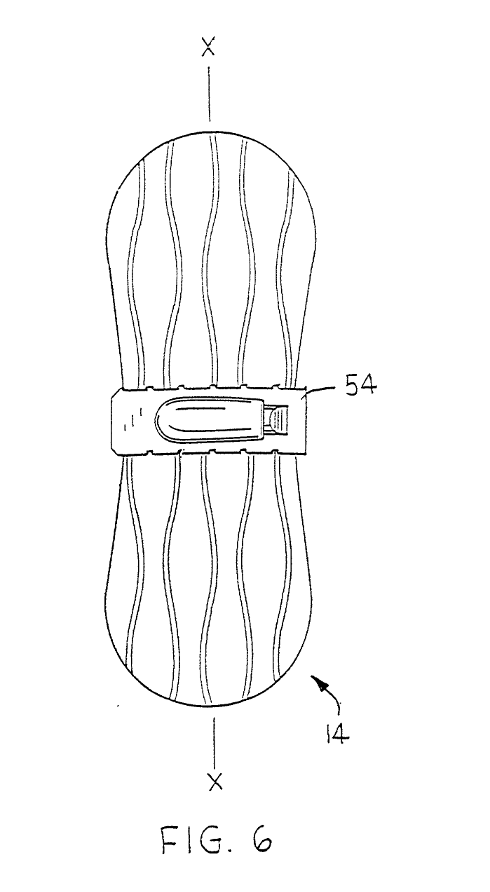

Referring now to FIG. 5, panty shield 14 is illustrated with a different vaginal insertion device, namely a tampon applicator tube 50 containing a tampon, only the withdrawal string 18 being illustrated, and a plunger 52. Both the tampon applicator tube 50 and plunger 52 are placed on panty shield 14 and oriented to central longitudinal axis in the same manner as tampon 16 in FIG. 1.

Referring to FIG. 6, another type of vaginal insertion device is illustrated in the form of a vaginal suppository 54 placed on panty shield 14 in a manner similar to that of tampon 16 in FIG. 1.

The intent of FIGS. 5 and 6 is to illustrate that various types of vaginal insertion devices can be associated with panty shield 14 and outer cover 12, so as to form a feminine sanitary protection device of the present invention.

Turning now primarily to FIGS. 3 and 4, the feminine sanitary protection device 10 of FIG. 1 is illustrated in a partially tri-folded manner. Although a tri-fold is desired, other folds are contemplated by the present invention, such as a C-fold. The panty shield (i.e., absorbent article or device) 14 and its integral outer cover 12 are folded together along fold lines 56, 58, thereby forming fold panel 60, fold panel 62, and base panel 64. As the tri-folding step or process continues, first joining element 32 joins portions of outer cover 12 together. Specifically, as fold panel 60 is fully folded downwardly, as viewed in FIG. 3, and fold panel 62 is fully folded downwardly, outer cover portion 66 of fold panel 60 is adhered to outer cover portion 70 of base panel 64, and outer cover portion 68 of fold panel 62 is adhered to outer cover portion 66 of fold panel 60. In a similar manner, second joining element 36 joins second side section 30, thereby producing the feminine sanitary protection device 10 with an integral pouch 13 formed of the outer cover 12 as illustrated in FIG. 4. In this final folded form illustrated in FIG. 4, first frangible line 34 is in generally overlapping alignment with itself, as illustrated in FIG. 4. In a similar manner, second frangible line 38 is also in generally overlapping alignment with itself. A peel strip 46 protecting the release strip adhesive can be seen on the exterior of the pouch.

It is contemplated that the pouch edge 47 may be secured by a dab or line of adhesive or cohesive material on the peel strip or on the interior surface of the outer cover 12 or even on an absorbent article component such as a bodyside liner or cover or both. In this context, it should be understood that one or more of the absorbent article components may cover the entire interior, inner or bodyside facing surface of the absorbent article. Alternatively, the absorbent article components may end just short of the edge of the outer cover 12 leaving a lip or rim of liquid impermeable material. The pouch edge 47 may also be secured by a strip of adhesive material such as tape.

In use, a user takes feminine sanitary protection device 10, illustrated in FIG. 4, grasps, for example, first side section 28, and separates or tears a portion of it from outer cover 12 along frangible line 34. Similarly, a portion of second side section 38 would then be removed from outer cover 12 along second frangible line 38. Since both joining elements 32, 36 (FIG. 1) are between their respective outer cover side edges 24, 26 and central longitudinal axis X-X, when side sections 28, 30 are removed, so are joining elements 32, 36, thereby permitting the outer cover 12 with its integral absorbent article (e.g., panty shield) 14 to be unfolded. The release strip 46 is removed from the outer cover 12, so that panty shield 14 (with the outer cover 12 forming as an integral liquid impermeable baffle) can be adhered to the undergarment.

Due to the tri-folded shape of panty shield 14, several advantages are created. One of these is the elimination of the curling tendency associated with those prior art napkins or panty shields that are rolled about a tampon or other similar device. Upon unfolding the tri-folded panty shield 14, it can be easily smoothed-out along fold lines 56, 58, thereby reassuming its desired shape or form, and allowing the panty shield to be easily adhered to an undergarment.

Another advantage with the present invention is that the tri-folded panty shield 14 does not diminish the comfort or absorbency in the Z-direction, which is associated with those earlier attempts with a rolled napkin or panty liner.

Still another advantage with the present invention is that the tri-folded panty shield 14 will not lose its embossing or other surface characteristics, which can occur with those earlier attempts utilizing a rolled napkin or panty liner.

Outer cover 12 can be made of any suitable material, and is desirably composed of a plastic material such as polyethylene or polypropylene. However, it may be composed of other materials such as polyethylene oxide (PEO), polyvinyl alcohol (PVOH), polycaporolactone (PCL), paper, or a nonwoven material such as a spunbond/meltblown material. Outer cover 12, as illustrated in FIG. 1, is larger in size than absorbent article (e.g., panty shield) 14. In a rectangular shape, outer cover 12 can have dimensions in the range, by way of example only, of about 10-25 centimeters (cm) in length and about 6-11 cm in width. This compares to an absorbent article (e.g., panty shield) 14 generally having dimensions of about 16 cm in length and about 5.5 cm in width. Since the dimensions of the absorbent article (e.g., panty shield) 14 can vary, outer cover 12 desirably has dimensions in the range of about 1-5 cm greater in length and in width than absorbent article (e.g., panty shield) 14. The purpose of the larger size of outer cover 12 is to provide the additional material for the tri-folding and joining of outer cover 12 illustrated in FIGS. 3 and 4, without undesirably affecting absorbent article (e.g., panty shield) 14 and the vaginal insertion device. Of course, when the side portions of the outer cover 12 are removed, the absorbent article (e.g., panty shield) and outer cover may be configured so the outer cover 12 has the same dimensions as the absorbent article.

One construction and material composition of a panty shield 14 is the absorbent pad described in U.S. Pat. No. 4,372,312, which issued Feb. 8, 1983; the contents of which are incorporated by reference herein. Another example of a panty shield is the absorbent pad described in U.S. Pat. No. 3,881,490, which issued on May 6, 1975, the contents of which are incorporated by reference herein. In these constructions, a separate liquid impermeable layer or baffle will be replaced by the outer cover 12 which is joined to the panty liner components (e.g., liquid permeable cover and absorbent medium) to function as a liquid impermeable layer or baffle when the device is unfolded. One example of the construction and material composition of a tampon is the tampon described in U.S. Pat. No. 5,807,372, which issued Sep. 15, 1998; the contents of which are incorporated by reference herein.

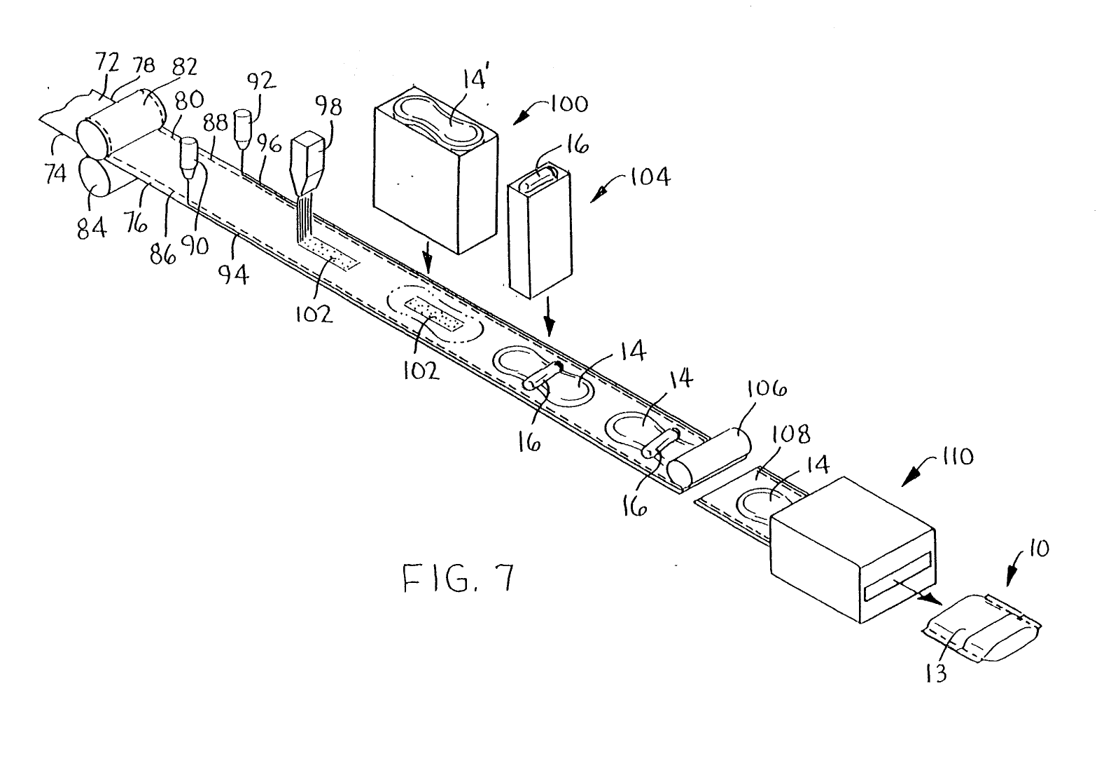

Referring now to FIG. 7, there is illustrated one exemplary method of making a feminine sanitary protection device in accordance with the principles of the present invention. As illustrated, a layer of material 72 continuously moves in a generally left-to-right direction as viewed in FIG. 7, and includes a side edge 74 and a side section 76 adjacent thereto, and a side edge 78 and a side section 80 adjacent thereto. The continuously moving layer of material 72 passes between a perforation roll 82 and an anvil roll 84 in order to provide a frangible line 86 in side section 76 and a frangible line 88 in side section 80. A pair of adhesive applicators 90, 92 apply an adhesive to continuously moving layer 72. Specifically, adhesive applicator 90 applies a joining element 94 between frangible line 86 and side edge 74, and adhesive applicator 92 applies joining element 96 between frangible line 88 and side edge 78. As layer 72 continues to move, it passes an adhesive applicator 98 that applies an adhesive, desirably intermittently, along or near the centerline of layer 72, and a panty shield component placement station 100 places a plurality of panty shield components 14′ (e.g., units formed by combining a liquid permeable liner and an absorbent medium) at spaced apart intervals upon discrete deposits of an adhesive 102 applied by an adhesive applicator 98 to form panty shields 14.

After the component placement station 100, a vaginal insertion device placement station 104 intermittently and selectively places a vaginal insertion device 16 on each panty shield 14. Thereafter, a die-cutting roll 106 forms a plurality of individual panty shields 14 and respective individual integral portions 108 of layer 72. Thereafter, a folding station 110 tri-folds each individual panty shield 14 and its respective integral portion 108 into a feminine sanitary protection device 10 with the integral portion 108 of layer 17 forming an integral pouch 13, as illustrated in both FIGS. 4 and 7.

According to the present invention, the above-described process can be modified in various ways. For example, the folding station 110 can include, in addition to a folding function, a pressing function in order to firmly adhere first side section 28 (FIG. 3) and second side section 30 of outer cover 12 firmly together. Further, folding station 110 can also include the function of the die-cutting roll 106. Such various types of apparatus are well known in the art in order to perform these cutting, folding, pressing, and other functions. Similarly, the perforation roll 82 and anvil roll 84 can be any suitable rolls or apparatus well known in the art in order to provide a line of selected perforations. The adhesive applicators 90, 92, 98 can be any suitable adhesive applicators well known in the art, and adhesive applicators 90,92 can apply adhesive in either a continuous or intermittent manner.

In another modification of the process illustrated in FIG. 7, the perforation roll 82 and anvil roll 84 can be a part of folding station 110 so that the forming of a frangible line can be accomplished after each individual panty shield 14 and its respective integral portion 108 of layer 72 is folded. In other words, integral portion 108 would not have any frangible lines 86, 88 prior to folding station 110, and after an integral portion 108 and its respective panty shield 14 are tri-folded, then folding station 110, or another suitable apparatus, can provide or form the frangible lines along the side sections of the tri-folded device. In this particular modification, the adhesive applicators 90, 92 may also be eliminated, and their joining function can be accomplished by means of the frangible lines 86, 88, in this example a plurality of perforations. Alternatively and/or additionally, the joining function may be accomplished by thermal bonding, ultrasonic bonding or other joining techniques.

In the above description of one aspect of the present invention, each panty shield 14 includes a separate release strip 46 (FIG. 2) for protecting the release strip adhesive 48 located on the outer cover 12, which in turn is used to secure the panty shield 14 (with integral outer cover 12 forming the liquid impermeable baffle) in an undergarment. The user opens the pouch 13 by removing a portion of the first side section 28 between the first frangible line 34 and the first side edge and a portion of the second side section 30 between the second frangible line 38 and the second side edge 26. The user then unfolds the panty shield 14 with its integral outer cover 12 which functions as the liquid impermeable layer and removes the release strip 46 to expose the exposes release strip adhesive 48 (FIG. 2) for attaching the panty shield 14 to an undergarment. Again, other similar modifications can be accomplished, including rearranging the sequence of steps or events in the method illustrated in FIG. 7.

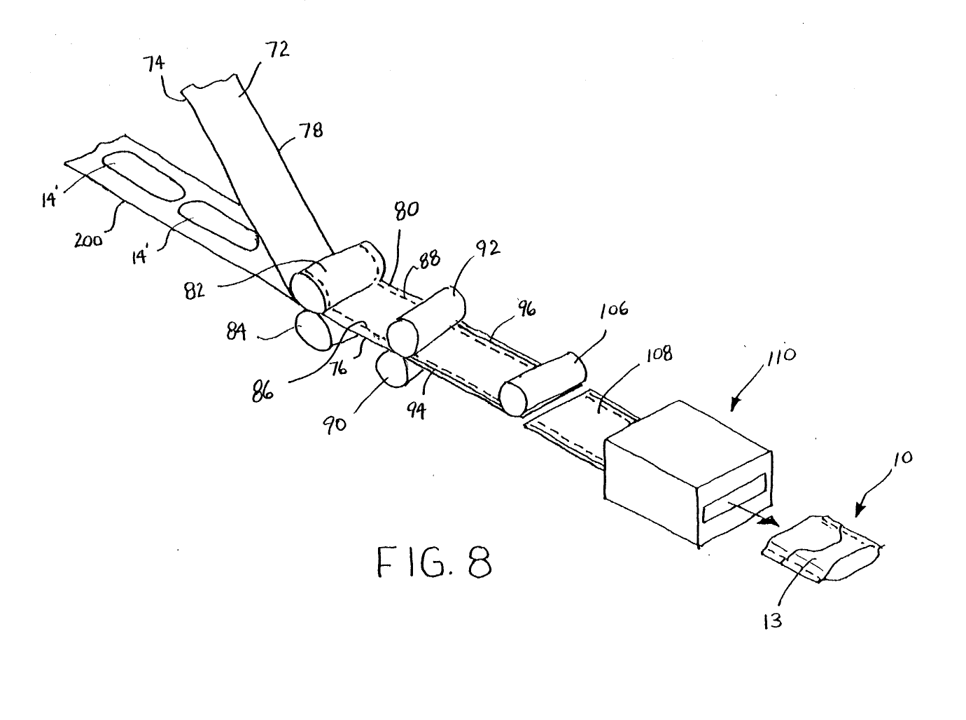

Referring now to FIG. 8, there is illustrated another exemplary method of making a feminine sanitary protection device in accordance with the principles of the present invention. As illustrated, a layer of material 72 continuously moves in a generally left-to-right direction as viewed in FIG. 8, and includes a side edge 74 and a side section 76 adjacent thereto, and a side edge 78 and a side section 80 adjacent thereto.

Absorbent article components 14′ are supported on a carrier sheet 200 at spaced apart intervals. The carrier sheet may be a nonwoven material that can function as the bodyside liner or cover of an absorbent article. These components 14′ are aligned with and joined to the layer of material 72 utilizing conventional adhesive, thermal bonding and/or ultrasonic bonding techniques to form absorbent articles. Optionally, a vaginal insertion device placement station may be used to intermittently and selectively places a vaginal insertion device on each absorbent article component prior to aligning with and joining to the layer of material.

The absorbent articles (not shown because they are covered by the continuously moving layer of material 72) pass between a perforation roll 82 and an anvil roll 84 in order to provide a frangible line 86 in side section 76 and a frangible line 88 in side section 80. A pair of adhesive applicator rolls 90, 92 apply an adhesive to one or both faces of the absorbent articles/continuously moving layer 72. Specifically, adhesive applicator rolls 90 and 92 apply a joining element 94 between frangible line 86 and side edge 74 and joining element 96 between frangible line 88 and side edge 78. Each joining element 94 and 96 may be on one or both faces (e.g., the top surface, the bottom surface or both surfaces) of the combined absorbent article/continuously moving layer 72.

Thereafter, a die-cutting roll 106 forms a plurality of individual panty shields 14 and respective individual integral portions 108 of layer 72. At this stage or immediately prior, release strip adhesive and a release strip (not shown) may be added to the layer 72. Thereafter, a folding station 110 tri-folds each individual absorbent article and its respective integral portion 108 into a feminine sanitary protection device 10 with the integral portion 108 of layer 17 forming an integral pouch 13, as illustrated in both FIGS. 4 and 7.

According to the present invention, the above-described process can be modified in various ways. For example, the folding station 110 can include, in addition to a folding function, a pressing function in order to firmly adhere first side section 28 (FIG. 3) and second side section 30 of outer cover 12 firmly together. Further, folding station 110 can also include the function of the die-cutting roll 106. Such various types of apparatus are well known in the art in order to perform these cutting, folding, pressing, and other functions. Similarly, the perforation roll 82 and anvil roll 84 can be any suitable rolls or apparatus well known in the art in order to provide a line of selected perforations. The adhesive applicator rolls 90, 92 can be any suitable adhesive applicators rolls well known in the art, and adhesive applicator rolls 90,92 can apply adhesive in either a continuous or intermittent manner.

In another modification of the process illustrated in FIG. 8, the perforation roll 82 and anvil roll 84 can be a part of folding station 110 so that the forming of a frangible line can be accomplished after each individual absorbent article and its respective integral portion 108 of layer 72 is folded. In other words, integral portion 108 would not have any frangible lines 86, 88 prior to folding station 1 10, and after an integral portion 108 and its respective absorbent article are tri-folded, then folding station 110, or another suitable apparatus, can provide or form the frangible lines along the side sections of the tri-folded device. In this particular modification, the adhesive applicator rolls 90, 92 may also be eliminated, and their joining function can be accomplished by means of the frangible lines 86, 88, in this example a plurality of perforations. Alternatively and/or additionally, the joining function may be accomplished by thermal bonding, ultrasonic bonding or other joining techniques.

Again, other similar modifications can be accomplished, including rearranging the sequence of steps or events in the method illustrated in FIG. 8.

In the above description of one aspect of the present invention, each absorbent article includes a separate release strip 46 (FIG. 2) for protecting the release strip adhesive 48 located on the outer cover 12, which in turn is used to secure the absorbent article in an undergarment.

While this invention has been described as having a preferred embodiment, it will be understood that it is capable of further modifications. It is therefore intended to cover any variations, equivalents, uses, or adaptations of the invention following the general principles thereof, and including such departures from the present disclosure as come or may come within known or customary practice in the art to which this invention pertains and falls within the limits of the appended claims.

What is claimed is:

1. A liquid absorbent device, comprising:
an outer cover having a central longitudinal axis, and including a top edge, a bottom edge, a first side edge and a second side edge, a first side section between the first side edge and the central longitudinal axis, and a second side section between the second the side edge and the central longitudinal axis,
a first frangible line in the first side section of the outer cover, and a second frangible line in the second side section of the outer cover,
a liquid permeable liner and an absorbent medium joined with the outer cover to form a liquid absorbent device, at least the liquid absorbent medium being located between the first frangible line and the second frangible line,
the liquid absorbent device being folded so the outer cover forms the exterior of the folded liquid absorbent device such that, upon removal of portions of the outer cover outboard the frangible lines and unfolding the liquid absorbent device, a remaining portion of the outer cover is adapted to form a liquid impermeable layer of the liquid absorbent device that includes the liquid permeable liner and the absorbent medium.

2. The device of claim 1 further comprising a first joining element in the first side section, and a second joining element in the second side section.

3. The device of claim 2 wherein the first joining element and the second joining element join respective portions of the folded outer cover together.

4. The device of claim 1 wherein each frangible line is in generally overlapping alignment with itself.

5. The device of claim 2 wherein the first frangible line is between the first joining element and the central longitudinal axis, and the second frangible line is between the second joining element and the central longitudinal axis.

6. The device of claim 1 further comprising a release strip for the liquid absorbent device.

7. The device of claim 5 further comprising a vaginal insertion device placed with the liquid absorbent device.

8. A feminine sanitary protection device, comprising:
an outer cover having a central longitudinal axis, and including a top edge, a bottom edge, a first side edge and a second side edge, a first side section between the first side edge and the central longitudinal axis, and a second side section between the second side edge and the central longitudinal axis,
means in the first side section and in the second side section for removing at least a portion of the first side section and at least a portion of the second side section from the outer cover, and
a liquid permeable liner and an absorbent medium joined with the outer cover to form a liquid absorbent device,
the liquid absorbent device being folded so the outer cover forms the exterior of the folded liquid absorbent device such that, upon removal of at least a portion of the first side section and at least a portion of the second side section from the outer cover and unfolding the liquid absorbent device, a remaining portion of the outer cover is adapted to form a liquid impermeable layer of the liquid absorbent device that includes the liquid permeable liner and the absorbent medium.

9. The device of claim 8 further comprising means in the first side section and in the second side section for joining respective portions of the folded outer cover together.

10. The device of claim 8 wherein the liquid absorbent device with its outer cover is tri-folded.

11. The device of claim 9 wherein the removing means is between the joining means.

12. The device of claim 8 further comprising a release strip for the liquid absorbent device.

13. The device of claim 8 wherein the removing means includes a first frangible line in the first side section, and a second frangible line in the second side section.

14. The device of claim 13 wherein each the frangible line includes perforations in the outer cover.

15. The device of claim 8 further comprising a vaginal insertion device placed with the liquid absorbent device.

16. A method of making a feminine sanitary protection device, comprising the steps of:
providing a continuously moving layer of liquid impermeable material including a pair of side edges and a pair of side portions;
aligning a plurality of absorbent article components at spaced apart intervals with the continuously moving layer of liquid impermeable material and joining the components to the liquid impermeable material to form absorbent articles;
forming a frangible line in each of the side portions;
applying a joining element in each of the side portions;
forming a plurality of individual absorbent articles with its respective individual portions of the layer of liquid impermeable material, and folding each individual absorbent article and its respective portion of the layer of material together.

17. The method of claim 16, wherein plurality of absorbent article components are supported on a carrier sheet that forms a bodyside cover or liner of the absorbent article.

18. The method of claim 16 further comprising the step of placing a vaginal insertion device on each absorbent article.

19. The method of claim 16 wherein each frangible line is formed between the joining element of its respective side section and a central longitudinal axis of the layer of material.

20. The method of claim 16 wherein each joining element is applied continuously.

21. The method of claim 16 wherein each joining element is applied intermittently.

22. The method of claim 16 wherein the step of forming a plurality of individual absorbent articles and respective individual portions of the layer of material includes cutting the layer of material between the panty shields.

23. The method of claim 16 wherein the step of folding includes tri-folding each absorbent article and its respective individual portion of the layer of liquid impermeable material.

24. A method of making a feminine sanitary protection device, comprising the steps of:
providing a continuously moving layer of liquid impermeable material including a pair of side edges and a pair of side sections,
forming a frangible line in each of the side sections of the layer of material,
applying a joining element in each of the side sections of the layer of material,
placing a plurality of panty shield components at spaced apart intervals on the continuously moving layer of liquid impermeable material and joining the components to the liquid impermeable layer to form panty shields,
forming a plurality of individual panty shields and respective individual portions of the layer of material, and
folding each individual portion of the layer of material and its panty shield together.

25. A method of making a feminine sanitary protection device, comprising the steps of:
providing a continuously moving layer of liquid impermeable material including a pair of side edges and a pair of side sections,
forming a frangible line in each of the side sections,
placing a plurality of panty shield components at spaced apart intervals on the continuously moving layer of liquid impermeable material and joining the components to the liquid impermeable layer to form panty shields,
forming a plurality of individual panty shields and respective individual portions of the layer of material, and
folding each individual portion of the layer of material and its panty shield together.

26. The method of claim 25 further comprising the step of applying a joining element in each of the side sections of the layer of material.

27. The method of claim 25 further comprising the step of placing a vaginal insertion device on each panty shield.

28. The method of claim 25 wherein each frangible line is formed between the joining element of its respective side section and a central longitudinal axis of the layer of material.
